(19) United States
(12) Patent Application Publication
(10) Pub. No.: US 2008/0262445 A1
(43) Pub. Date: Oct. 23, 2008

(54) Transdermal Delivery of Hydrophobic Bioactive Agents

(75) Inventors: Tsung-Min Hsu, Minneapolis, MN (US); Alan T.J. Hickey, Eden Prairie, MN (US); Eric C. Jacobson, San Diego, CA (US); Nicole T. Gricenko, St. Louis Park, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: DERMATRENDS, INC., Minneapolis, MN (US)

(21) Appl. No.: 11/574,928

(22) PCT Filed: Sep. 8, 2005
(86) PCT No.: PCT/US05/31825

§ 371 Date: Jun. 6, 2008

Related U.S. Application Data
(60) Provisional application No. 60/607,922, filed on Sep. 8, 2004.

(51) Int. Cl. A61F 13/02; A61K 31/5513; A61K 31/5517; A61K 31/56

(52) U.S. Cl. 604/307; 514/221; 514/220; 514/169; 424/449

(57) ABSTRACT

A method and related compositions, including the use of N-acyl derivatives of sarcosine, provide for the delivery of bioactive agents through tissue surfaces such as the skin. The method and composition are particularly well suited for hydrophobic active agents such as serotonin (5HT₃) receptor antagonists (e.g., ondansetron), antipsychotic agents (e.g., risperidone), benzodiazepines (e.g., flumazenil), and progestins (e.g., levonorgestrel).

### TECHNICAL FIELD

The present invention relates to transdermal drug delivery systems. In another aspect, the invention relates to the delivery of hydrophobic drugs through the skin or other tissue surfaces tissues.

### BACKGROUND OF THE INVENTION

The transdermal delivery of drugs remains an evolving and promising area of medical treatment. Unfortunately, as of today, only a small number of drugs have been successfully commercialized in transdermal form. See, for example, “Current Status and Future Potential of Transdermal Drug Delivery”, M R Prausnitz, et al., Nature Reviews 3:115-124 (February 2004). The authors of this article conclude that “[d]espite these successes, the number of drugs that can be administered using conventional patches is very limited. Still, the authors remain optimistic and conclude that “although individual chemical enhancers have had limited success, combinations of chemical enhancers offer new opportunities in transdermal formulations”. Still, this article and others in the art confirms that there are few commercial products currently on the market that meet the requirements demanded of such a formulation, in terms of effectiveness, stability, comparability, safety, ease of use, and cost.

On a separate subject, various aspects regarding the use of N-acyl derivatives of sarcosine in contact with the skin has been described previously. See, for instance, “Breaking the Skin Barrier”, Nature Reviews: Drug Discovery, Vol. 3, page 112 (February 2004), which summarizes a variety of skin patch formulations, including one containing N-lauroyl sarcosine:sorbitan monolaurate 20. See also, R S Lanigan, Int J Toxicol. 2001:20 Suppl 1:1-14 (abstract), which mentions in part that “[t]hese ingredients are nonirritating and nonsensitizing to animal and human skin, although they can enhance the penetration of other ingredients through the skin. For that reason, caution should be exhibited in formulating cosmetic products that contain these ingredients in combination with other ingredients whose safety is based on their lack of absorption or where dermal absorption is a concern (e.g., HC Yellow No. 4, Disperse Yellow 3).”

On a separate subject, hydrophobic drugs are known to be particularly difficult to deliver transdermally. See, for instance, web-based literature provided by Acusphere, Inc. (http://www.acusphere.com/hydrophob.html), which describes the manner in which “many hydrophobic drugs are comprised of particles that are relatively large and therefore have a limited surface area available for interaction with water. These hydrophobic drugs are often formulated in less than ideal ways in order to make them dissolve. It is possible to increase the dissolution rate of hydrophobic drugs by increasing their aggregate surface area.” To accomplish this, the literature goes on to describe how various processes have been attempted, including micronization, which entails grinding hydrophobic drugs into smaller microparticles, or the use of oils like Cremophor, in order to dissolve the drugs, or the attempt formulate such hydrophobic drugs can be formulated into soft gelatin capsules, but these are only suitable for oral administration and encapsulate only a small volume of drug.

Finally, various patents and other references purport to describe the transdermal delivery of specific drugs or classes. See, for instance, European patent application EP 0879051B1, for “Rate controlled Transdermal Administration of Risperidone”.

Applicant's themselves have previously described transdermal delivery systems that include, inter alias the use of hydroxide-releasing agents as skin permeation enhancers. See, for example, U.S. Pat. No. 6,586,000, the disclosure of which is incorporated herein by reference.

Still, and in spite of considerable progress in the development of new formulations for transdermal delivery, there remain several bioactive agents for which transdermal delivery might be desired, but for which an effective composition has not yet been provided in commercial form.

### SUMMARY OF THE INVENTION

The present invention provides a method and apparatus for enhancing the flux of a drug through a body surface, the method comprising the step of administering the drug to a localized region of a human patient's body surface in combination with a solubilizing enhancer system that comprises:

a) one or more N acyl derivatives of sarcosine,

b) one or more compatible agents adapted to contribute to the solubilization of the bioactive agent in the composition and/or to its enhanced permeation across a tissue barrier such as the skin, ingredients (a) and (b) being present total and relative amounts effective to both solubilize and enhance the flux of the bioactive agent through the localized region of the body surface in an amount sufficient to achieve a therapeutic effect. Optionally, and preferably, the composition also includes a pressure adhesive in combination an inert powder sufficient to provide physical and structural integrity to the resulting patch.

In a preferred embodiment, the bioactive agent comprises a hydrophobic drug selected from the group consisting of specific serotonin (5HT₃) receptor antagonists (e.g., ondansetron), antipsychotic agents (e.g., risperidone), benzodiazepines (e.g., flumazenil), and a progestin (e.g., levonorgestrel) present in an amount adapted to provide a desired therapeutic effect; the N-acyl derivatives of sarcosine comprise N-lauroyl sarcosine, present in an amount between about 0.1 and about 10 percent, by weight based on the dry weight of the composition, and the one or more compatible solubilizing/enhancing comprise a combination of one or more polyols, and more preferably alkylene glycols, present in an amount between about 3 and about 30 percent, in combination with one or more tocopherols (such as Vitamin E), present in an amount between about 3 and about 30 percent.

A composition of the present invention can be prepared in any suitable manner and form. In a preferred embodiment, for instance, a bioactive agent, such as a water insoluble compound, is first dissolved in one or more organic solubilizers such as vitamin E, PGML or hexylene glycol, after which n-lauroyl sarcosine is then dissolved in order to form a stable composition.

N-lauroyl sarcosine has been suggested for use as an enhancer itself. However, at skin temperature, about 32° C., it does not have the solubilizing properties for water insoluble compounds. To overcome this solubility issue, Applicants have found that the inclusion of one or more additional ingredients, such as vitamin E and PGML/or hexylene glycol, can be used to both improve the solubility of the bioactive agent. More surprisingly, when combined with n-lauroyl sarcosine, the resulting composition has been found to enhances the bioactive agent's permeation through human skin and maintains this permeation through multiple days.

PGML and hexylene glycol can also be used as skin enhancers in their own right. Without the n-lauroyl sarcosine, however, the rate of permeation through the skin is not as good as the combination of n-lauroyl sarcosine, vitamin E and PGML/or hexylene glycol.

Optionally, and preferably, the composition is prepared in the form of a drug delivery system, e.g., a topical or transdermal “patch,” wherein the active agent is contained within a laminated structure that is to be affixed to the skin. In such a structure, the drug composition is contained in a layer, or “reservoir,” underlying an upper backing layer. The laminated structure may contain a single reservoir, or it may contain multiple reservoirs. In a particularly preferred embodiment, the reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the active agent, composition, and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene).

Preferred compositions of this invention are capable of delivering a hydrophobic drug in a therapeutic manner, e.g., at a rate of about 50 mg/day, preferably 20 mg/day, more preferably 10 mg/day, most preferably 5 mg/day.

### DETAILED DESCRIPTION

The method and system of the present invention provide a composition adapted to enhance bioactive agent permeation through human skin. The composition, in turn, comprises an N-acyl derivative of sarcosine, such as n-lauroyl sarcosine, in combination with one or more co-enhancers such as an alkylene glycol such as propylene glycol monolaurate (PGML), and preferably also including a tocopherol such as vitamin E.

Suitable sarcosines provide a desired combination of properties such as biocompatibility, as well as compatability with the other enhancer/solubilzing agents, and with the bioactive agent as well.

Examples of suitable N-acyl derivatives of sarcosine are generally referred to as acyl sarcosines, as well as those that are salts, known generally as acyl sarcosinates. Preferred sarcosine derivatives are selected from the group of fatty acids that appear in these acyl sarcosines and sarcosinates (Coconut Acid, Oleic Acid, Lauric Acid, and Myristic Acid). In each case the fatty acid has been determined to be either safe for use or safe as used in cosmetic formulations. See, for instance, R S Lanigan, Int J Toxicol. 2001:20 Suppl 1:1-14 (abstract), which states:

Acyl sarcosines are considered modified fatty acids with greater solubility and increased acidity of the carboxylic acid group compared to the parent fatty acid. They are used in a large number of cosmetic formulations as hair-conditioning agents and surfactant-cleansing agents. In soaps, concentrations are reported to be as high as 12.9%. These ingredients have low oral toxicity in rats. Although cytotoxic to Chinese hamster cells in culture, acyl sarcosines and sarcosinates are not mutagenic in those cells, nor in bacterial cells in culture. Carcinogenicity data were not available. These ingredients are nonirritating and nonsensitizing to animal and human skin, although they can enhance the penetration of other ingredients through the skin. For that reason, caution should be exhibited in formulating cosmetic products that contain these ingredients in combination with other ingredients whose safety is based on their lack of absorption or where dermal absorption is a concern (e.g., HC Yellow No. 4, Disperse Yellow 3). Because sarcosine can be nitrosated to form N-nitrososarcosine, a known animal carcinogen, these ingredients should not be used in cosmetic products in which N-nitroso compounds may be formed. With the above caveat, and based on the available data, it was concluded that these acyl sarcosines and sarcosinates are safe as used in rinse-off products. They may be safely used in leave-on products at concentrations up to 5%, the highest concentration tested in clinical irritation and sensitization studies. Oleoyl Sarcosine is used as a corrosion inhibitor in some aerosol products, at extremely low concentrations. In this circumstance, the ingredient is not being used as a cosmetic ingredient and this report is not intended to limit that use. Because of the absence of data on inhalation toxicity, however, it was concluded that the available data were not sufficient.

Suitable N-lauroyl sarcosines can be obtained commercially and from a variety of sources, for example, from Sigma Aldrich Chemical. Suitable examples include N-acyl sarcosines [N-oleoyl sarcosine (CAS Reg. No. 110-25-8); N-stearoyl sarcosine (CAS Reg. No. 142-48-3); N-lauroyl sarcosine (CAS Reg. No. 97-78-9); N-myristoyl sarcosine (CAS Reg. No. 52558-73-3); N-cocoyl sarcosine mixture (CAS Reg. No. 68411-97-2); and sodium N-acyl sarcosinates [N-methyl-N-(1-oxo-9-octodecenyl) glycine (CAS Reg. No. 3624-77-9); N-methyl-N-(1-oxooctadecyl) glycine (CAS Reg. No. 5136-55-0); N-methyl-N-(1-oxododecyl) glycine (CAS Reg. No. 137-16-6); N-methyl-N-(1-oxotetradecyl glycine (CAS Reg. No. 30364-51-3); and N-cocoyl sarcosine sodium salt mixture (CAS Reg. No. 61791-59-1)].

Suitable alkylene glycols provide an optimal combination of such properties as biocompatibility, cost, compatability with the sarcosinate(s) of choice, and the ability to contribute to either the solubility and/or permeation of the bioactive agent across a tissue barrier such as the skin.

Examples of suitable alkylene glycols include, but are not limited to ethylene and propylene glycols, and are described, for instance, in pp. 566-568, the disclosure of which is incorporated herein by reference. Preferred alkylene glycols are selected from the group consisting of mono-, di-, and triglycols. Suitable alkylene glycols can be obtained commercially and from a variety of sources, for example, from Sigma Aldrich.

Suitable tocopherols are those providing an optimal combination of such properties as biocompatibility and the ability to solublize the bioactive agent and/or enhance its permeation across a tissue barrier such as the skin. Examples of suitable tocopherols include alpha-tocopherol and alpha-tocopherol acetate. Preferred tocopherols are commercially available, for instance, from Sigma Aldrich

For those drugs having an unusually low rate of permeation through the skin or mucosal tissue, it may be desirable to include one or more additional permeation enhancers. Suitable secondary enhancers (or “co-enhancers”) include, but are not limited to, ethers such as diethylene glycol monoethyl ether (available commercially as Transcutol) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450; see also ); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; sulfoxides such as DMSO and N-decylmethyl sulfoxide (C10MSO) may also be used, but are less preferred. Percutaneous Penetration Enhancers, eds. Smith et al. (CRC Press, 1995) provides an excellent overview of the field and further information concerning possible secondary enhancers for use in conjunction with the present invention.

The active agent administered may be any compound that is suitable for topical, transdermal or transmucosal delivery and induces a desired local or systemic effect. Such substances include the broad classes of compounds normally delivered through body surfaces and membranes, including skin. The amount of active agent administered will depend on a number of factors and will vary from subject to subject and depend on the particular drug administered, the particular disorder or condition being treated, the severity of the symptoms, the subject's age, weight and general condition, and the judgment of the prescribing physician. Other factors, specific to transdermal drug delivery, include the solubility and permeability of the carrier and adhesive layer in a drug delivery device, if one is used, and the period of time for which such a device will be fixed to the skin or other body surface. The minimum amount of drug is determined by the requirement that sufficient quantities of drug must be present in a device or composition to maintain the desired rate of release over the given period of application. The maximum amount for safety purposes is determined by the requirement that the quantity of drug present cannot exceed a rate of release that reaches toxic levels. Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties.

Among the hydrophobic drugs which may be formulated in accordance with the present invention may be mentioned the following:

Analgesics and anti-inflammatory agents: aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac.

Anthelmintics: albendazole, bephenium hydroxynaphthoate, cambendazole, dichlorophen, ivermectin, mebendazole, oxamniquine, oxfendazole, oxantel embonate, praziquantel, pyrantel embonate, thiabendazole.

Anti-arrhythmic agents: amiodarone HCl, disopyramide, flecainide acetate, quinidine sulphate. Anti-bacterial agents: benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim.

Anti-coagulants: dicoumarol, dipyridamole, nicoumalone, phenindione.

Anti-depressants: amoxapine, maprotiline HCl, mianserin HCL, nortriptyline HCl, trazodone HCL, trimipramine maleate.

Anti-diabetics: acetohexamide, chlorpropamide, glibenclamide, gliclazide, glipizide, tolazamide, tolbutamide.

Anti-epileptics: beclamide, carbamazepine, clonazepam, ethotoin, methoin, metlisuximide, methylphenobarbitone, oxcarbazepine, paramethadione, phenacemide, phenobarbitone, phenytoin, phensuximide, primidone, sulthiame, valproic acid.

Anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid.

Anti-gout agents: allopurinol, probenecid, sulphin-pyrazone.

Anti-hypertensive agents: amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL.

Anti-malarials: amodiaquine, chloroquine, chlorproguanil HCl, halofantrine HCl, mefloquine HCl, proguanil HCl, pyrimethamine, quinine sulphate.

Anti-migraine agents: dihydroergotamine mesylate, ergotamine tartrate, methysergide maleate, pizotifen maleate, sumatriptan succinate.

Anti-muscarinic agents: atropine, benzhexol HCl, biperiden, ethopropazine HCl, hyoscyamine, mepenzolate bromide, oxyphencylcimine HCl, tropicamide.

Anti-neoplastic agents and Immunosuppressants: aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone.

Anti-protazoal agents: benznidazole, clioquinol, decoquinate, diiodohydroxyquinoline, diloxanide furoate, dinitolmide, furzolidone, metronidazole, nimorazole, nitrofurazone, omidazole, tinidazole.

Anti-thyroid agents: carbimazole, propylthiouracil.

Anxiolytic, sedatives, hypnotics and neuroleptics: alprazolam, amylobarbitone, barbitone, bentazepam, bromazepam, bromperidol, brotizolam, butobarbitone, carbromal, chlordiazepoxide, chlornethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lonnetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone.

Beta.-blockers: acebutolol, alprenolol, atenolol, labetalol, metoprolol, nadolol, oxprenolol, pindolol, propranolol.

Cardiac Inotropic agents: amrinone, digitoxin, digoxin, enoximone, lanatoside C, medigoxin.

Corticosteroids: beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone.

Diuretics: acetazolamide, amiloride, bendrofluazide, bumetanide, chlorothiazide, chlorthalidone, ethacrynic acid, frusemide, metolazone, spironolactone, triamterene.

Anti-parkinsonian agents: bromocriptine mesylate, lysuride maleate.

Gastro-intestinal agents: bisacodyl, cimetidine, cisapride, diphenoxylate HCl, domperidone, famnotidine, loperamide, mesalazine, nizatidine, omeprazole, ondansetron HCL, ranitidine HCl, sulphasalazine.

Histamine H,-Receptor Antagonists: acrivastine, astemizole, cinnarizine, cyclizine, cyproheptadine HCl, dimenhydrinate, flunarizine HCl, loratadine, meclozine HCl, oxatomide, terfenadine.

Lipid regulating agents: bezafibrate, clofibrate, fenofibrate, gemfibrozil, probucol.

Nitrates and other anti-anginal agents: amyl nitrate, glyceryl trinitrate, isosorbide dinitrate, isosorbide mononitrate, pentaerythritol tetranitrate.

Nutritional agents: betacarotene, vitamin A, vitamin B.sub.2, vitamin D, vitamin E, vitamin K.

Opioid analgesics: codeine, dextropropyoxyphene, diamorphine, dihydrocodeine, meptazinol, methadone, morphine, nalbuphine, pentazocine.

Sex hormones: clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone.

Stimulants: amphetamine, dexamphetamine, dexfenfluramine, fenfluramine, mazindol.

Mixtures of hydrophobic drugs may, of course, be used where therapeutically effective. The concentration of drug in the final pharmaceutical formulation will be that which is required to provide the desired therapeutic effect from the drug concerned, but generally will lie in the range 0.1% to 50% by weight, based on the weight of the final composition. However, in many instances the present compositions will have better bioavailability than known compositions of the drug concerned, whereby the drug concentration may be reduced as compared with the conventional preparations without loss of therapeutic effect.

Ondansetron, represents a particularly preferred form of serotonin (5HT₃) receptor antagonists, and in turn, is the approved name for 1,2,3,9-tetrahydro-9-methyl-3-[(2-methyl-1H-imidazol-1-yl)methyl]-4H-carba zol-4-one, is a highly selective and potent antagonist of 5-hydroxytryptamine (5-HT) at 5-HT.sub.3 recaptots. Ondansetron, together with its physiologically acceptable salts and solvetea, is described and claimed in British Patent No. 2153821B, and may be used in the treatment of a variety of conditions, including the nausea and vomiting induced by cancer chemotherapy and radiotherapy (as described, for example, in European Patent Specification No. 226266A).

The preferred form of ondansetron for pharmaceutical formulation is the hydrochloride dihydrate. Ondansetron hydrochloride dihydrate may be presented in a variety of formulations, one of which is as tablets for oral administration, when particularly suitable unit doses of the drug substance for the treatment of emesis are 5 mg and 10 mg.

Risperidone is an antipsychotic agent belonging to a new chemical class, the benzisoxazole derivatives. The chemical designation is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one. U.S. Pat. Nos. 4,804,663 and 6,750,341, the contents of which are incorporated by reference, which describe the synthesis of risperidone, while the preparation and pharmacological activity thereof are described in EP-0,196,132. The term risperidone as used herein comprises the free base form and the pharmaceutically acceptable acid addition salts thereof. The solubility of risperidone is increased upon the formation of such salt forms, which can be obtained by reaction of the base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic, maleic, fumaric, malic, tartaric, citric, methane-sulfonic, ethanesulfonic, benzenesulfonic, p-toluenesulfonic, cyclamic, salicylic, p-aminosalicylic, pamoic and the like acids. The term addition salt as used hereinabove also comprises the solvates which risperidone as well as the salts thereof, are able to form. Such solvates are for example hydrates, alcoholates and the like.

The amount of risperidone in the present compositions ranges from 0.01% to 1%, preferably from 0.02% to 0.5%, and most preferably from 0.05% to 0.2%.

Suitable estrogens that may be administered using the compositions and drug delivery systems of the invention include synthetic and natural estrogens such as: estradiol (i.e., 1,3,5-estratriene-3,17.beta.-diol, or “17.beta.-estradiol”) and its esters, including estradiol benzoate, valerate, cypionate, heptanoate, decanoate, acetate and diacetate; 17.alpha.-estradiol; ethinylestradiol (i.e., 17.alpha.-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. 17.beta.-Estradiol, ethinylestradiol and mestranol are particularly preferred synthetic estrogenic agents for use in conjunction with the present invention.

Suitable progestins that can be delivered using the compositions and systems of the invention include, but are not limited to, acetoxypregnenolone, allylestrenol, anagestone acetate, chlormadinone acetate, cyproterone, cyproterone acetate, desogestrel, dihydrogesterone, dimethisterone, ethisterone (17.alpha.-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. Progesterone, medroxyprogesterone, norethindrone, norethynodrel, d,1-norgestrel and 1-norgestrel are particularly preferred progestins.

It is generally desirable to co-administer a progestin along with an estrogen in female HRT so that the estrogen is not “unopposed.” As is well known, estrogen-based therapies are known to increase the risk of endometrial hyperplasia and cancer, as well as the risk of breast cancer, in treated individuals. Co-administration of estrogenic agents with a progestin has been found to decrease the aforementioned risks. Preferred such combinations include, without limitation: 17.beta.-estradiol and medroxyprogesterone acetate; 17.beta.-estradiol and norethindrone; 17.beta.-estradiol and norethynodrel; ethinyl estradiol and d,1-norgestrel; ethinyl estradiol and 1-norgestrel; and megestrol and medroxyprogesterone acetate.

For female HRT, it may be desirable to co-administer a small amount of an androgenic agent along with the progestin and the estrogen, in order to reproduce the complete hormone profile of the premenopausal woman, since low levels of certain androgens are present in premenopausal women. Any of the aforementioned steroid drugs may be naturally occurring steroids, synthetic steroids, or derivatives thereof. Administration of a combination of steroidal active agents is useful in a variety of contexts, as will be readily appreciated by those skilled in the art. For example, the transdermal administration of a progestin with an estrogen may be used in female hormone replacement therapy, so that the symptoms or conditions resulting from altered hormone levels is mitigated or substantially prevented. The present compositions and drug delivery systems are in addition useful to administer progestins and estrogens to treat other conditions and disorders that are responsive to transdermal administration of the combination of active agents. For example, the aforementioned combination is useful to treat the symptoms of premenstrual stress and for female contraception, as noted above. For female hormone replacement therapy, the woman undergoing treatment will generally be of childbearing age or older, in whom ovarian estrogen, progesterone and androgen production has been interrupted either because of natural menopause, surgical procedures, radiation, chemical ovarian ablation or extirpation, or premature ovarian failure. For hormone replacement therapy, and for the other indications described herein including female contraception, the compositions or drug delivery systems are preferably used consecutively so that administration of the active agents is substantially continuous. Transdermal drug administration according to the invention provides highly effective female hormone replacement therapy. That is, the incidence and severity of hot flashes and night sweats are reduced, postmenopausal loss of calcium from bone is minimized, the risk of death from ischemic heart disease is reduced, and the vascularity and health of the Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties. However, preferred transdermal compositions and systems for hormone replacement therapy are capable of delivering about 0.5 to 10.0 mg progestin, e.g., norethindrone, norethindrone acetate or the like, and about 10 to 200 .mu.g estrogen, e.g., 17.beta.-estradiol, ethinyl estradiol, mestranol or the like, over a period of about 24 hours. However, it will be appreciated by those skilled in the art that the desired dose of each individual active agent will depend on the specific active agent as well as on other factors; the minimum effective dose of each active agent is of course preferred

Flumazenil (flumazepil, Anexate®, Lanexat®, Mazicon®, Romazicon®) is a benzodiazepine antagonist, used as an antidote in the treatment of benzodiazepine overdose. Its chemical description is ethyl 8-fluoro-5,6-dihydro-5-methyl-6-oxo-4H-imidazo[1,5-a][1,4]benzodiazepine-3-carboxylate. The drug reverses the effects of benzodiazepines by competitive inhibition of benzodiazepine receptors. The onset of action is very fast, about one to two minutes. The activity peak is six to ten minutes. Many benzodiazepines have longer half-lives than flumazenil. Therefore repeat doses of flumazenil may be required to prevent recurrent symptoms of overdosage once the initial dose of flumazenil wears off. It was introduced in 1987 by Hoffinann-La Roche under trade name Anexate.

The method of delivery of the active agent may vary, but necessarily involves application of a formulation or drug delivery system containing a composition of the present invention to a predetermined area of the skin or other tissue for a period of time sufficient to provide the desired local or systemic effect. The method may involve direct application of the composition as an ointment, gel, cream, or the like, or may involve use of a drug delivery device.

Suitable formulations include ointments, creams, gels, lotions, pastes, and the like. Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol.RTM. trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions, which are preferred for delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethyl-ammonium (DOTMA) liposomes are available under the tradename Lipofectin.RTM. (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. They are generally although not necessarily formed from lipids, preferably charged lipids such as phospholipids. Preparation of lipidic microspheres is well known in the art and described in the pertinent texts and literature.

Various additives, known to those skilled in the art, may be included in the topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulation may also contain irritation-mitigating additives to minimize or eliminate the possibility of skin irritation or skin damage resulting from the drug, the enhancer, or other components of the formulation. Suitable irritation-mitigating additives include, for example: .alpha.-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the formulations.

The concentration of the active agent in the formulation can vary a great deal, and will depend on a variety of factors, including the disease or condition to be treated, the nature and activity of the active agent, the desired effect, possible adverse reactions, the ability and speed of the active agent to reach its intended target, and other factors within the particular knowledge of the patient and physician. Preferred formulations will typically contain on the order of about 0.5 wt. % to 50 wt. %, optimally about 10 wt. % to 30 wt. %, active agent.

An alternative and preferred method involves the use of a drug delivery system, e.g., a topical or transdermal “patch,” wherein the active agent is contained within a laminated structure that is to be affixed to the skin. In such a structure, the drug composition is contained in a layer, or “reservoir,” underlying an upper backing layer. The laminated structure may contain a single reservoir, or it may contain multiple reservoirs.

In one embodiment, the reservoir comprises a polymeric matrix of a pharmaceutically acceptable adhesive material that serves to affix the system to the skin during drug delivery; typically, the adhesive material is a pressure-sensitive adhesive (PSA) that is suitable for long-tenn skin contact, and which should be physically and chemically compatible with the active agent, composition, and any carriers, vehicles or other additives that are present. Examples of suitable adhesive materials include, but are not limited to, the following: polyethylenes; polysiloxanes; polyisobutylenes; polyacrylates; polyacrylamides; polyarethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing drug or other composition components. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure includes a release liner. Immediately prior to use, this layer is removed from the device so that the system may be affixed to the skin. The release liner should be made from a drug/vehicle impermeable material, and is a disposable element which serves only to protect the device prior to application. Typically, the release liner is formed from a material impermeable to the pharmacologically active agent and composition, and which is easily stripped from the transdermal patch prior to use.

In an alternative embodiment, the drug-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device.

A rate-controlling membrane, if present, will be included in the system on the skin side of one or more of the drug reservoirs. The materials used to form such a membrane are selected to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5 cm.sup.2 to 200 cm.sup.2, preferably 5 cm.sup.2 to 100 cm.sup.2, more preferably 20 cm.sup.2 to 60 cm.sup.2. That area will vary, of course, with the amount of drug to be delivered and the flux of the drug through the body surface. Larger patches will necessary to accommodate larger quantities of drug, while smaller patches can be used for smaller quantities of drug and/or drugs that exhibit a relatively high permeation rate.

Such drug delivery systems may be fabricated using conventional coating and laminating techniques known in the art. For example, adhesive matrix systems can be prepared by casting a fluid admixture of adhesive, drug and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/vehicle mixture. In general, transdermal systems of the invention are fabricated by solvent evaporation, film casting, melt extrusion, thin film lamination, die cutting, or the like. The composition of this invention will generally be incorporated into the device during patch manufacture rather than subsequent to preparation of the device.

In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating such adhesive overlayer, the delivery system remains in place for the required period of time.

Other types and configurations of transdermal drug delivery systems may also be used in conjunction with the method of the present invention, as will be appreciated by those skilled in the art of transdermal drug delivery. See, for example, Ghosh, Transdermal and Topical Drug Delivery Systems (Interpharm Press, 1997), particularly Chapters 2 and 8.

As with the topically applied formulations of the invention, the composition of this invention within the drug reservoir(s) of these laminated system may contain a number of components. In some cases, the drug and composition may be delivered “neat,” i.e., in the absence of additional liquid. In most cases, however, the drug will be dissolved, dispersed or suspended in a suitable pharmaceutically acceptable vehicle, typically a solvent or gel. Other components that may be present include preservatives, stabilizers, surfactants, and the like. The invention accordingly provides a novel and highly effective means for increasing the flux of an active agent through the body surface (skin or mucosal tissue) of a human or animal.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains. Furthermore, the practice of the present invention will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 9th Ed. (New York: McGraw-Hill, 1996).

All patents, patent applications, publications and other references cited herein are incorporated by reference in their entireties.

The following examples are put forth so as to provide those of ordinary skill in the art with a complete disclosure and description of how to make and use the compounds of the invention, and are not intended to limit the scope of what the inventors regard as their invention. Efforts have been made to ensure accuracy with respect to numbers (e.g., amounts, temperature, etc.) but some errors.

### EXAMPLES

### Example 1

Ondansetron Permeation

An in vitro skin permeation study was conducted using one ondansetron transdermal patch. The formulations used to prepare these systems are listed in Table 1, which includes weight and percent weight of each component of the dried formulations. Each component was added in the order listed in Table 1. “PVPP’ refers to a commercially available polyvinyl polypyrrolidone powder, which was added in an amount sufficient to balance the liquid nature of other solubizing agents in order to maintain the physical integrity of the patch. Other suitable and generally inert powders that can be used will become apparent to those skilled in the art, given the present description. “Duratak” is a tradename and refers to a commercially available polyisobutylene adhesive liquid available from National Starch and Chemical.

Each formulation was coated on a release liner and dried in an oven at 65° C. for two hours to remove water and other solvents. The dried drug-in-adhesive/release liner film was laminated to a backing film. The backing/drug-in-adhesive/release liner laminate was then cut into discs with a diameter of 9/16 inch.

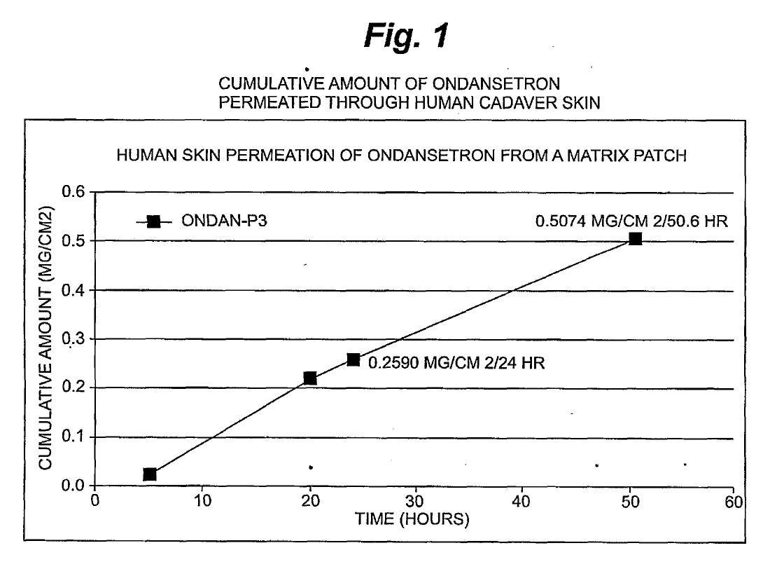

The in vitro permeation of ondansetron through human cadaver skin from these discs was performed using Franz diffusion cells with a diffusion area of 1 cm² and a receiver solution capacity of 8 ml. Human cadaver skin was cut to a proper size and placed on a flat surface with the stratum corneum side facing up. The release liner was peeled away from the disc laminate. The backing/drug-in-adhesive film was placed and pressed on the skin with the adhesive side facing the stratum corneum. The skin/adhesive/backing laminate was clamped between the donor and receiver chambers of the diffusion cell with the skin side facing the receiver solution. Three diffusion cells were used for each formulation. The receiver solution was 1% (2-Hydroxypropyl)-□-cyclodextrin in 0.05M KH2PO4, pH 7.4. The entire receiver solution was collected and replaced with fresh receiver solution at each time point. The receiver solution collected was analyzed by HPLC to determine the concentration of ondansetron. The cumulative amount of ondansetron that permeated across the human cadaver skin was calculated using the measured ondansetron concentrations in the receiver solutions, which were plotted versus time and shown in FIG. 1. The cumulative amount of ondansetron that permeated through the skin was 0.26 mg/cm² after 24 hours and 0.51 mg/cm² after 51 hours. Sodium hydroxide is added as a pH modifier, not as an enhancer. Final pH of the patch is 4.7.

### Example 2

Risperidone Permeation

An in vitro skin permeation study was conducted using five risperidone transdermal patches. The formulations used to prepare these systems are listed in Table 2, which includes weight and percent weight of each component of the dried formulations. Each component was added in the order listed in Table 2. Each formulation was coated on a release liner and dried in an oven at 65° C. for two hours to remove water and other solvents. The dried drug-in-adhesive/release liner film was laminated to a backing film. The backing/drug-in-adhesive/release liner laminate was then cut into discs with a diameter of 9/16 inch.

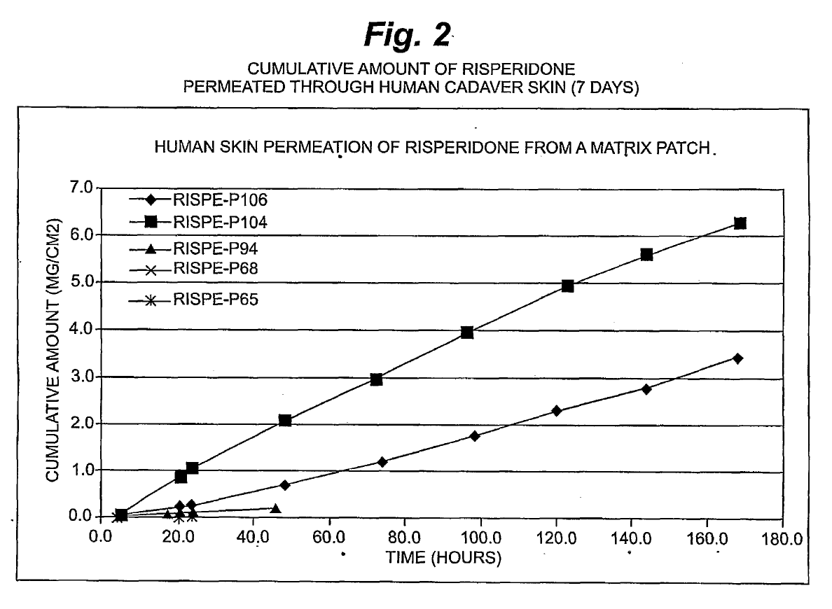

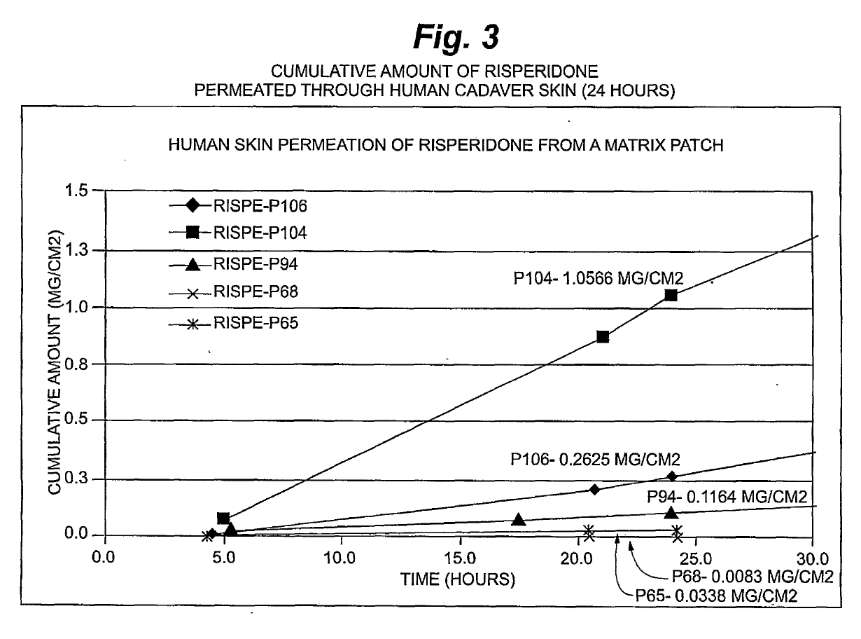

The in vitro permeation of risperidone through human cadaver skin from these discs was performed using Franz diffusion cells with a diffusion area of 1 cm² and a receiver solution capacity of 8 ml. Human cadaver skin was cut to a proper size and placed on a flat surface with the stratum corneum side facing up. The release liner was peeled away from the disc laminate. The backing/drug-in-adhesive film was placed and pressed on the skin with the adhesive side facing the stratum corneum. The skin/adhesive/backing laminate was clamped between the donor and receiver chambers of the diffusion cell with the skin side facing the receiver solution. Three diffusion cells were used for each formulation. The receiver solution was 1% (2-Hydroxypropyl)-□-cyclodextrin in 0.05M KH2PO4, pH 7.4. The entire receiver solution was collected and replaced with fresh receiver solution at each time point. The receiver solution collected was analyzed by HPLC to determine the concentration of risperidone. The cumulative amount of risperidone that permeated across the human cadaver skin was calculated using the measured risperidone concentrations in the receiver solutions, which were plotted versus time and shown in FIGS. 2 & 3.

N-lauroyl sarcosine was added to the compositions of Rispe-P65, P68 and P94. In each case, the patch pH was above 9.5 (10.90, 10.18 and 9.80 respectively). The addition of sodium hydroxide does offer a certain degree of skin permeation with this bioactive agent. For instance, the cumulative amount of risperidone that permeated through the skin with Rispe-P94 was 0.12 mg/cm²/24 hr. When n-lauroyl sarcosine was added, the cumulative amount of risperidone that permeated through the skin was 1.06 mg/cm² after 24 hours (with Rispe-P104), which was about 9.8 times higher than when no n-lauroyl sarcosine was present in the formulation. This permeation was maintained over a seven-day period. The cumulative amount of risperidone that permeated through the skin after this period was 6.3 mg/cm². Rispe-P106 is an example of a composition containing n-lauroyl sarcosine in combination with vitamin E and hexlyene glycol. In this case, the cumulative amount of risperidone that permeated through the skin was 0.26 mg/cm² after 24 hours and 3.42 mg/cm² after seven days. The final patch pH of Rispe-P104 and P106 were 7.44 and 7.94 respectively.

### Example 3

Levonorgestrel Permeation

An in vitro skin permeation study was conducted using four levonorgestrel transdermal patches. The formulations used to prepare these systems are listed in Table 3, which includes weight and percent weight of each component of the dried formulations. Each component was added in the order listed in Table 3. Each formulation was coated on a release liner and dried in an oven at 65° C. for two hours to remove water and other solvents. The dried drug-in-adhesive/release liner film was laminated to a backing film. The backing/drug-in-adhesive/release liner laminate was then cut into discs with a diameter of 9/16 inch.

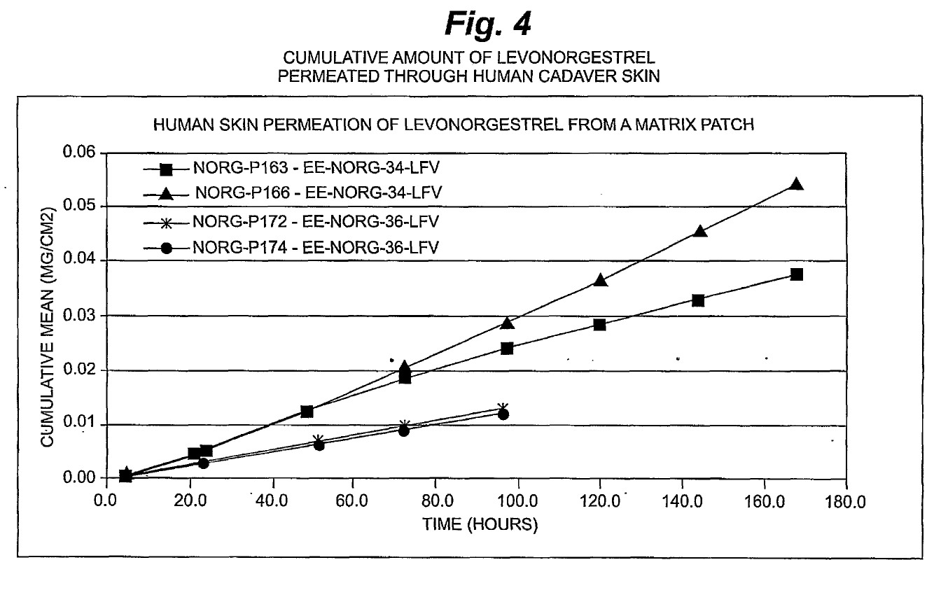

The in vitro permeation of levonorgestrel through human cadaver skin from these discs was performed using Franz diffusion cells with a diffusion area of 1 cm² and a receiver solution capacity of 8 ml. Human cadaver skin was cut to a proper size and placed on a flat surface with the stratum corneum side facing up. The release liner was peeled away from the disc laminate. The backing/drug-in-adhesive film was placed and pressed on the skin with the adhesive side facing the stratum corneum. The skin/adhesive/backing laminate was clamped between the donor and receiver chambers of the diffusion cell with the skin side facing the receiver solution. Three diffusion cells were used for each formulation. The receiver solution was 1% (2-Hydroxypropyl)-□-cyclodextrin in 0.05M KH2PO4, pH 7.4. The entire receiver solution was collected and replaced with fresh receiver solution at each time point. The receiver solution collected was analyzed by HPLC to determine the concentration of levonorgestrel. The cumulative amount of levonorgestrel that permeated across the human cadaver skin was calculated using the measured levonorgestrel concentrations in the receiver solutions, which were plotted versus time and shown in FIG. 4.

No sodium lauroyl sarcosine was added to the compositions of Norg-P172 and P174. The cumulative amount of levonorgestrel that permeated through the skin with Norg-P172 was 0.0031 mg/cm²/23.3 hr. When sodium lauroyl sarcosine was added, the cumulative amount of levonorgestrel that permeated through the skin was 0.005 mg/cm² after 24 hours (with Norg-P166), which was about 1.6 times higher than when no sodium lauroyl sarcosine was present in the formulation. This permeation was maintained over a seven-day period. The cumulative amount of levonorgestrel that permeated through the skin after this period was 0.0543 mg/cm². Norg-P163 is an example of a composition containing sodium lauroyl sarcosine in combination with vitamin E and PGML. In this case, the cumulative amount of levonorgestrel that permeated through the skin was 0.0050 mg/cm² after 24 hours and 0.0375 mg/cm² after seven days.

### Example 4

Flumazenil Permeation

An in vitro skin permeation study was conducted using two flumazenil transdermal patches. The formulations used to prepare these systems are listed in Table 4, which includes weight and percent weight of each component of the dried formulations. Each component was added in the order listed in Table 4. Each formulation was coated on a release liner and dried in an oven at 65° C. for two hours to remove water and other solvents. The dried drug-in-adhesive/release liner film was laminated to a backing film. The backing/drug-in-adhesive/release liner laminate was then cut into discs with a diameter of 9/16 inch.

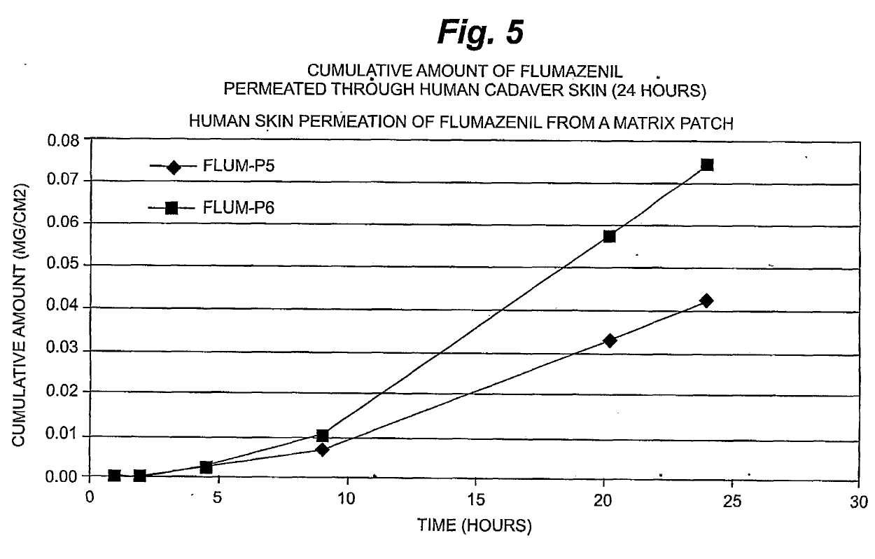

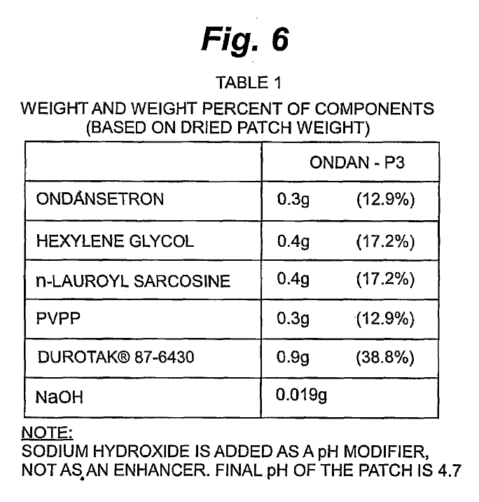

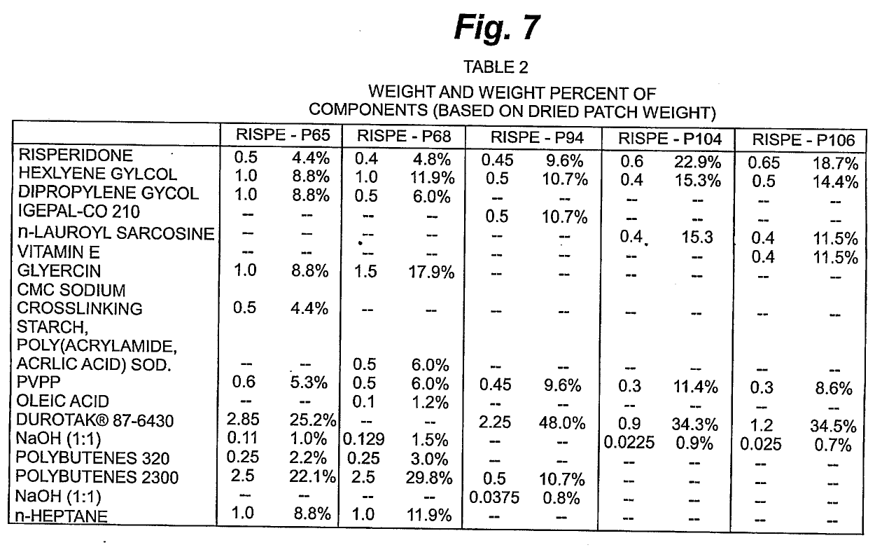

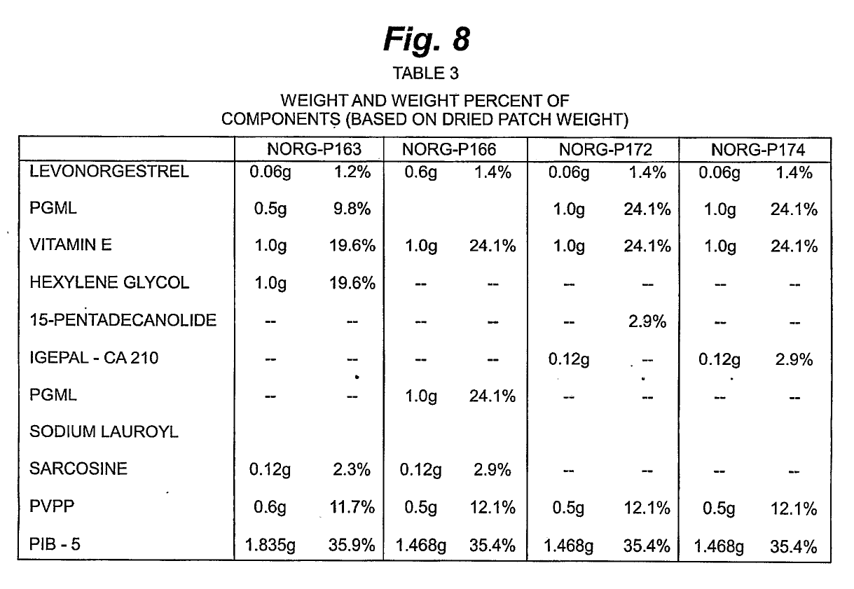

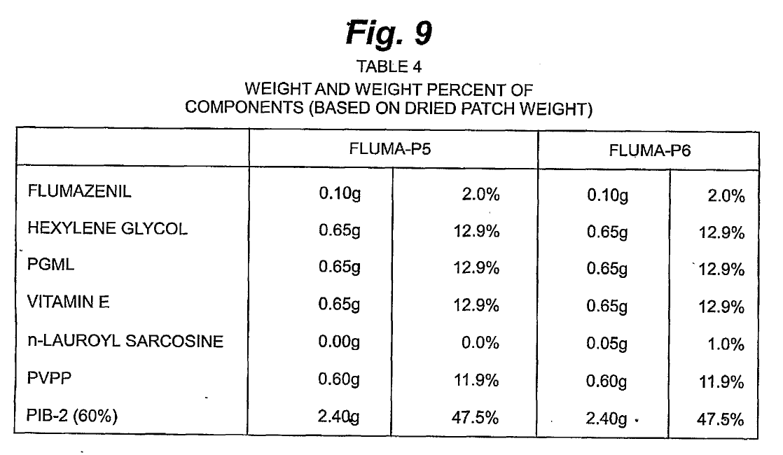

The in vitro permeation of flumazenil through human cadaver skin from these discs was performed using Franz diffusion cells with a diffusion area of 1 cm² and a receiver solution capacity of 8 ml. Human cadaver skin was cut to a proper size and placed on a flat surface with the stratum corneum side facing up. The release liner was peeled away from the disc laminate. The backing/drug-in-adhesive film was placed and pressed on the skin with the adhesive side facing the stratum corneum. The skin/adhesive/backing laminate was clamped between the donor and receiver chambers of the diffusion cell with the skin side facing the receiver solution. Three diffusion cells were used for each formulation. The receiver solution was 1% (2-Hydroxypropyl)-□-cyclodextrin in 0.05M KH2PO4, pH 7.4. The entire receiver solution was collected and replaced with fresh receiver solution at each time point. The receiver solution collected was analyzed by HPLC to determine the concentration of flumazenil. The cumulative amount of flumazenil that permeated across the human cadaver skin was calculated using the measured flumazenil concentrations in the receiver solutions, which were plotted versus time and shown in FIG. 5.

A certain degree of skin permeation with this bioactive agent was found with Fluma-P5. The cumulative amount of flumazenil that permeated through the skin was 0.042 mg/cm²/24 hr. When n-lauroyl sarcosine was added (Fluma-P6), the cumulative amount of flumazenil that permeated through the skin was 0.074 mg/cm² after 24 hours, which was about 1.76 times higher than when no n-lauroyl sarcosine was present in the formulation.

What is claimed is:

1. A method for enhancing the rate at which a hydrophobic active agent can be administered in stable form to a patient's body surface in order to permeate into and/or through the body surface, the method comprising providing a composition that comprises a hydrophobic active agent in combination with a) one or more N acyl derivatives of sarcosine, and b) one or more compatible agents adapted to contribute to the solubilization of the bioactive agent in the composition and/or to its enhanced permeation across a tissue barrier such as the skin, wherein ingredients (a) and (b) are present in total and relative amounts effective to both solubilize and enhance the flux of the bioactive agent through the localized region of the body surface in an amount sufficient to achieve a therapeutic effect.

2. A method according to claim 1 wherein the bioactive agent comprises a hydrophobic drug selected from the group consisting of specific serotonin (5HT₃) receptor antagonists, antipsychotic agents, benzodiazepines, and progestins.

3. A method according to claim 2 wherein the N-acyl derivative of sarcosine comprises N-lauroyl sarcosine.

4. A method according to claim 3 wherein the N-lauroyl sarcosine is present in an amount between about 0.1 and about 10 percent, by weight based on the dry weight of the composition.

5. A method according to claim 1 wherein the one or more compatible solubilizing/enhancing comprises a combination of one or more polyols in combination with one or more tocopherols.

6. A method according to claim 5 wherein the polyols are present in an amount between about 3 and about 30 percent, and the one or more tocopherols are present in an amount between about 3 and about 30 percent.

7. A method according to claim 6 wherein the N-acyl derivative of sarcosine comprises N-lauroyl sarcosine.

8. A method according to claim 7 wherein the N-lauroyl sarcosine is present in an amount between about 0.1 and about 10 percent, by weight based on the dry weight of the composition.

9. A method according to claim 8 wherein the bioactive agent comprises a hydrophobic drug selected from the group consisting of specific serotonin (5HT₃) receptor antagonists, antipsychotic agents, benzodiazepines, and progestins.

10. A method according to claim 9 wherein the specific serotonin (5HT₃) receptor antagonists comprise ondansetron, the antipsychotic agents comprise risperidone, the benzodiazepines comprise flumazenil, and the progestin comprises levonorgestrel.

11. A composition for enhancing the rate at which a hydrophobic active agent can be administered in stable form to a patient's body surface in order to permeate into and/or through the body surface, the composition comprising a hydrophobic active agent in combination with a) one or more N acyl derivatives of sarcosine, and b) one or more compatible agents adapted to contribute to the solubilization of the bioactive agent in the composition and/or to its enhanced permeation across a tissue barrier such as the skin, wherein ingredients (a) and (b) are present in total and relative amounts effective to both solubilize and enhance the flux of the bioactive agent through the localized region of the body surface in an amount sufficient to achieve a therapeutic effect.

12. A composition according to claim 11 wherein the bioactive agent comprises a hydrophobic drug selected from the group consisting of specific serotonin (5HT₃) receptor antagonists, antipsychotic agents, benzodiazepines, and progestins.

13. A composition according to claim 12 wherein the N-acyl derivative of sarcosine comprises N-lauroyl sarcosine present in an amount between about 0.1 and about 10 percent, by weight based on the dry weight of the composition.

14. A composition according to claim 11 wherein the one or more compatible solubilizing/enhancing comprises one or more polyols present in an amount between about 3 and about 30 percent, and one or more tocopherols are present in an amount between about 3 and about 30 percent.

15. A composition according to claim 11 wherein the bioactive agent comprises a hydrophobic drug selected from the group consisting of specific serotonin (5HT₃) receptor antagonists, antipsychotic agents, benzodiazepines, and progestins; the N-acyl derivative of sarcosine comprises N-lauroyl sarcosine present in an amount between about 0.1 and about 10 percent, by weight based on the dry weight of the composition; and the one or more compatible solubilizing/enhancing comprises one or more polyols present in an amount between about 3 and about 30 percent, and one or more tocopherols are present in an amount between about 3 and about 30 percent.

16. A composition according to claim 15 wherein the specific serotonin (5HT₃) receptor antagonists comprise ondansetron, the antipsychotic agents comprise risperidone, the beizodiazepines comprise flumazenil, and the progestin comprises levonorgestrel.

17. A drug delivery system comprising a composition according to any preceding claim.

18. A drug delivery system according to claim 17, comprising a topical or transdermal patch having the hydrophobic drug contained within a laminated structure that is to be affixed to the skin.

19. A drug delivery system according to claim 18 wherein the laminated structure comprises one or more reservoirs containing the composition, and further comprises a polymeric matrix of a pharmaceutically acceptable adhesive material that serves to affix the system to the skin during drug delivery.

20. A drug delivery system comprising a transdermal patch comprising a composition for enhancing the rate at which a hydrophobic active agent selected from the group consisting of ondansetron, risperidone, flumazenil, and levonorgestrel can be administered in stable form to a patient's body surface in order to permeate into and/or through the body surface, the composition comprising a hydrophobic active agent in combination with a) N-lauroyl sarcosine present in an amount between about 0.1 and about 10 percent, by weight based on the dry weight of the composition, and b) compatible solubilizing/enhancing agents comprising one or more polyols present in an amount between about 3 and about 30 percent, and one or more tocopherols are present in an amount between about 3 and about 30 percent.
